(19) United States
(12) Patent Application Publication
(10) Pub. No.: US 2024/0153643 A1
(43) Pub. Date: May 9, 2024

(54) INFORMATION PROCESSING APPARATUS, ESTIMATION METHOD AND PROGRAM

(71) Applicant: NIPPON TELEGRAPH AND TELEPHONE CORPORATION, Tokyo (JP)

(72) Inventors: Yuka NISHIDA, Tokyo (JP); Hideaki KIN, Tokyo (JP); Takeshi KURASHIMA, Tokyo (JP); Hiroyuki TODA, Tokyo (JP)

(21) Appl. No.: 18/548,756

(22) PCT Filed: Mar. 25, 2021
(86) PCT No.: PCT/JP2021/012577

§ 371 Date: Sep. 1, 2023

(51) Int. Cl. G16H 50/30

(52) U.S. Cl. CPC G16H 50/30

(57) ABSTRACT

An information processing apparatus includes: a parameter estimation unit that estimates, as parameters indicating a temporal change in interest of a user, a first parameter, a second parameter, and a third parameter based on data including a past behavior of the user and an evaluation value of the behavior, the first parameter being a parameter indicating inherent constant interest of the user, the second parameter being a parameter indicating an effect that the user is influenced by a past behavior and is attracted to a behavior option, and the third parameter being a parameter indicating an effect that the user loses interest due to boredom by a past behavior; a social welfare calculation unit that calculates a value indicating social welfare of the user based on the estimated parameters; and an optimum behavior selection unit that selects an optimum behavior of the user based on the calculated value indicating social welfare and outputs data indicating the selected optimum behavior.

### TECHNICAL FIELD

The present invention relates to an information processing apparatus, an estimation method, and a program.

### BACKGROUND ART

Techniques that estimate temporal changes in interest of a person, and estimate an optimum behavior based on a similarity between a tendency of the interest of the person and a behavior option are known. Changes in a current interest of the person are influenced every moment by a past behavior history. By presenting options for a future behavior that matches interest, the person can choose the options to enhance social welfare. The behavior options include products to be purchased next, movies to be viewed, and exercises to be performed as healthy behaviors.

For example, Non-Patent Document 1 describes temporal changes in interest of a user using the following three types of effects. The first involves inherent constant interest (inherent) of the user, the second is based on an effect (attraction) of being attracted by a behavior option, which is influenced by a past behavior, and the third involves an effect (aversion) of losing interest due to boredom of a past behavior. In Non-Patent Document 1, the behavior option is treated as having a continuous change without being tagged, and “a similarity between a tendency of interest of a person and a behavior option” is estimated as defined as social welfare of a person, in consideration of temporal changes in interest of the user.

Further, Non-Patent Document 2 indicates that, when the person continues similar behaviors, interest of the person is first attracted by the behaviors, and then is gradually lost. In Non-Patent Document 2, the social welfare of the person is estimated in consideration of the situation described above. In this case, the behavior option is treated as a tagged option.

### RELATED-ART DOCUMENT

### Non-Patent Document

Non-Patent Document 1: Wei Lu, Stratis Ioannidis & Lask V. S. Lakshmanan, “Optimal Recommendations under Attraction, Aversion, and Social Influence”, KDD '14: Proceedings of the 20th ACM SIGKDD international conference on Knowledge discovery and data mining, pp. 811-820 (2014)
Non-Patent Document 2: Romain Warlop, Alessandro Lazaric, Jeremie Mary, “Fighting Boredom in Recommender Systems with Linear Reinforcement Learning”, NeurIPS '18: Proceedings of Neural Information Processing Systems, (2018)

### SUMMARY OF INVENTION

### Technical Problem

In the technique disclosed in Non-Patent Document 1, when creating a model of the temporal changes in interest of the user, the behavior option is free of a tag, and it is assumed that the magnitude relationship between attraction and aversion is constant for each user. However, this is inconsistent with the technique disclosed in Non-Patent Document 2. In the technique disclosed in Non-Patent Document 2, the magnitude relationship between attraction and aversion is treated as changes with time. However, in the technique disclosed in Non-Patent Document 2, the behavior option is treated as a tagged option, which results in the issue of not using an option for a continuously changing behavior.

An object of a disclosed technique is to present a behavior option that matches a temporal change in interest of a user.

### Solution to Problem

According to a disclosed technique, there is provided an information processing apparatus including: a parameter estimation unit that estimates, as parameters indicating a temporal change in interest of a user, a first parameter, a second parameter, and a third parameter based on data including a past behavior of the user and an evaluation value of the behavior, the first parameter being a parameter indicating inherent constant interest of the user, the second parameter being a parameter indicating an effect that the user is influenced by a past behavior and is attracted to a behavior option, and the third parameter being a parameter indicating an effect that the user loses interest due to boredom by a past behavior; a social welfare calculation unit that calculates a value indicating social welfare of the user based on the estimated parameters; and an optimum behavior selection unit that selects an optimum behavior of the user based on the calculated value indicating the social welfare and outputs data indicating the selected optimum behavior, in which the effect indicated by the second parameter is larger than the effect indicated by the third parameter in a case where a selection frequency of the behavior option is low in the past behaviors of the user, and is smaller than the effect indicated by the third parameter in a case where a selection frequency of the behavior option is high in the past behaviors of the user.

### Advantageous Effects of Invention

It is possible to present a behavior option that matches a temporal change in interest of a user.

### BRIEF DESCRIPTION OF DRAWINGS

FIG. 1 is a functional configuration diagram of an information processing apparatus.

FIG. 2 is a flowchart illustrating an example of a flow of estimation processing.

FIG. 3 is a diagram illustrating a hardware configuration example of the information processing apparatus.

### DESCRIPTION OF EMBODIMENTS

Hereinafter, one or more embodiments (present embodiments) of the present invention will be described with reference to the drawings. The embodiments described below are merely an example, and embodiments to which the present invention is applied is not limited to the embodiments described below.

With use of behavior data for the user, an information processing apparatus according to the present embodiment estimates parameters related to a magnitude relationship between attraction and aversion, which are factors for interest of a user. In addition, the information processing apparatus estimates, based on the estimated parameters, a value indicating social welfare that involves a similarity between a tendency of the interest of the user and a behavior option, then selects the behavior option that maximizes the social welfare, and subsequently outputs data indicating the selected behavior option.

### (Functional Configuration of Information Processing Apparatus)

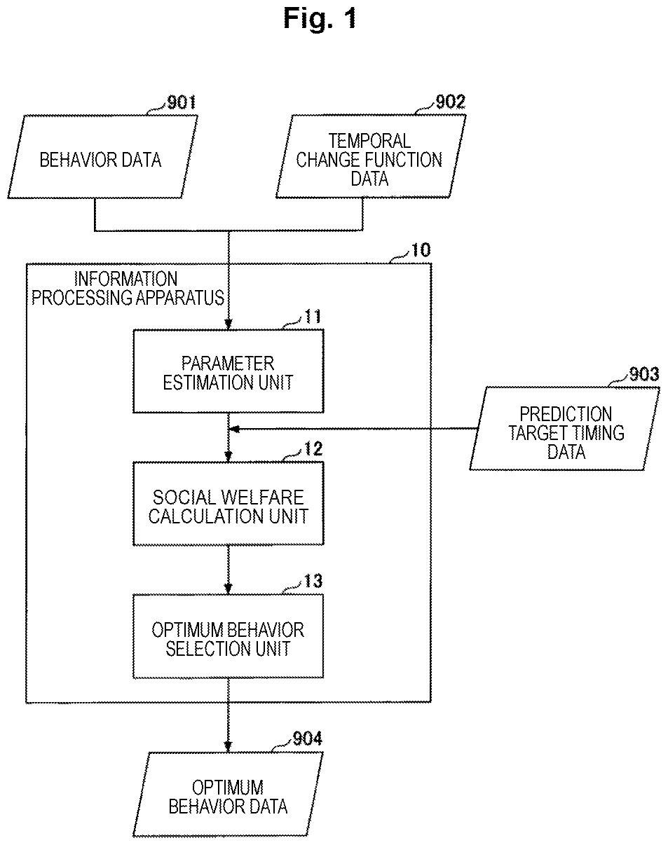

FIG. 1 is a diagram illustrating a functional configuration of the information processing apparatus according to the present embodiment. An information processing apparatus 10 includes a parameter estimation unit 11, a social welfare calculation unit 12, and an optimum behavior selection unit 13.

The parameter estimation unit 11 estimates parameters, based on behavior data 901 and time-varying function data 902.

The behavior data 901 is data including a past behavior of the user and an evaluation value of the behavior. Specifically, the behavior data 901 includes an evaluation value for a behavior of each user, and includes data of a time at which evaluation is performed, and the behavior data 901 is comprised of a user ID (denoted i), a behavior ID (denoted by j), the evaluation value for the behavior (denoted by r), and a time at which evaluation is performed (denoted by t). The behavior includes, for example, movie watching.

The time-varying function data 902 is data that defines a function representative of a model representing a temporal change in the interest of the user. In addition, the estimated parameters are parameters included in the function representative of the model. Specifically, Equations (1) and (2) below are illustrated as an example of a function representative of a model in which, for a temporal change uᵢ(t) of interest of a user i, attraction first becomes dominant, and then aversion gradually becomes dominant. In each model, v(t) that is a function indicative of a type of the behavior is used.

$$\begin{matrix}[ Math . 1 ] & \\ {u}_{i} ( t ) = {α}_{i} {u}_{i}^{0} + {γ}_{i} \frac{{\sum }_{τ = 0}^{t - 1} {e}^{- {ω}_{γ i} ( t - 1 - τ )} v ( τ )}{{\sum }_{τ = 0}^{t - 1} {{e}^{- {ω}_{γ i}}}^{( t - 1 - τ )}} - {δ}_{i} \frac{{\sum }_{τ = 0}^{t - 1} {e}^{- {ω}_{δ i} ( t - 1 - τ )} v ( τ )}{{\sum }_{τ = 0}^{t - 1} {e}^{- {ω}_{δ i} ( t - 1 - τ )}} & ( 1 )\end{matrix}$$

$$\begin{matrix}[ Math . 2 ] & \\ {u}_{i} ( t ) = {α}_{i} {u}_{i}^{0} + {γ}_{i} σ ( {N}_{i *} - {N}_{i} ) g ( {v}_{i} ( t - 1 ) ) {δ}_{i} σ ( {N}_{i} - {N}_{i *} ) g ( {v}_{i} ( t - 1 ) ) & ( 2 )\end{matrix}$$

Here, the followings are given.

$$σ ( {N}_{i *} - {N}_{i} ) = \frac{1}{1 + {e}^{- ( {N}_{i *} - {N}_{i} )}} \qquad [ Math . 3 ]$$

$${N}_{i} = v ( t - 1 ) \cdot g ( {v}_{i} ( t - 1 ) ) \qquad [ Math . 4 ]$$

$$g ( {v}_{i} ( t - 1 ) ) = \frac{{\sum }_{τ = 0}^{t - 1} {e}^{- {ω}_{i} ( t - 1 - τ )} v ( τ )}{{\sum }_{τ = 0}^{t - 1} {e}^{- {ω}_{i} ( t - 1 - τ )}} \qquad [ Math . 5 ]$$

Equation (1) includes five types of parameters, i.e., αᵢ, γᵢ, and δᵢ, indicating percentages of inherent, attraction, and aversion, and ω_{γi} and ω_{δi}, indicating forgetting rates of behaviors related to the attraction and the aversion. αᵢ is an example of a first parameter indicating inherent constant interest of the user. γᵢ is an example of a second parameter indicating an effect of attracting a behavior option that is influenced by a past behavior. δᵢ is an example of a third parameter indicating an effect of losing interest due to boredom of the past behavior.

The effect indicated by the second parameter is greater than the effect indicated by the third parameter if a selection frequency of the behavior option is low for past behaviors of the user, and if the selection frequency of the behavior option is high for past behaviors of the user.

Equation (2) includes five types of parameters, i.e., αᵢ, γᵢ, and δᵢ as denoted in Equation (1), a point N_{i*} at which a magnitude relationship between attraction and aversion is reversed, and (i indicating a forgetting rate of the behavior.

When using a function given by Equation (1), that is, a model that represents a temporal change in the interest of the user and uses weighs that are attenuated with time to calculate a weighted average for a past behavior history, the parameter estimation unit 11 estimates parameters that include the second parameter indicating a weight, and includes the third parameter that indicates a weight and is different from the second parameter. An advantage of the function given by Equation (1) is that a plurality of patterns for interest can be expressed based on a magnitude relation for the parameters.

When using the function given by Equation (2), that is, a model that represents a temporal change in the interest of the user, and calculates a weighted average for a past behavior history, the parameter estimation unit 11 estimates a parameter indicating a selection frequency in a case where the effect indicated by the third parameter becomes greater than the effect indicated by the second parameter. An advantage of the function given by Equation (2) is that, in addition to the advantage of Equation (1), by calculating and evaluating a similarity between a past behavior history and a latest behavior history, the number of similar behaviors until aversion for each user becomes dominant can be identified.

The parameter estimation unit 11 estimates the five types of parameters in Equation (1) or Equation (2), based on the behavior data 901 and the time-varying function data 902. Specifically, the parameter estimation unit 11 uses, as a similarity between a tendency of interest of the user and a type of the behavior, an inner product of a tendency uᵢ(t) of the interest of the user and a type v(t) of the behavior, to estimate uᵢ and vⱼ that minimize an error between the similarity and an evaluation for the behavior.

More specifically, the parameter estimation unit 11 performs matrix decomposition using a stochastic gradient descent. That is, the parameter estimation unit 11 estimates parameters uᵢ, vⱼ, λ, and μ that minimize the following Equation by using cross validation.

$${{min}_{{u}_{i} , {v}_{j} ,}}_{i ∈ [ n ] , j ∈ [ m ]} {\sum }_{( i , j ) ∈ E} {( {r}_{ij} - 〈 {u}_{i} , {v}_{j} 〉 )}^{2} + λ {\sum }_{i ∈ [ n ]} {‖ {u}_{i} ‖}^{2} + μ {\sum }_{j ∈ [ m ]} {‖ {v}_{j} ‖}^{2} \qquad [ Math . 6 ]$$

Here, n is the number of users, and m is the number of behavior options.

Subsequently, the parameter estimation unit 11 sets uᵢ⁰=uᵢ for the estimated parameters uᵢ, vⱼ, λ, and μ, and then estimates uᵢ(t) that minimizes the following equation.

$${{min}_{{u}_{i} ( t ) , i ∈ [ n ] , ,}}_{t ∈ [ T ]} {{\sum }_{t ∈ [ T ] , i ∈ [ n ] ,}}_{( i , j ) ∈ {E}_{i} ( t )} {( {r}_{ij} - 〈 {u}_{i} ( t ) , {v}_{j} 〉 )}^{2} + {\sum }_{i ∈ [ n ] , t ∈ [ T ]} ( {‖ {u}_{i} ( t ) - {u}_{i , model} ( t ) ‖}^{2} + κ {‖ {u}_{i} ( t ) ‖}^{2} ) \qquad [ Math . 7 ]$$

Here, T expresses a prediction target period, and indicates an elapsed time from Jan. 1, 1970 as a time stamp. In the present embodiment, an initial time is set to 0, and T expresses an elapsed time from the initial time. In addition, u_{i,model}(t) is a time-varying function that is given by Equation (1) or Equation (2) described above. uᵢ(t) depends on the parameters αᵢ, γᵢ, δᵢ, ω_{γi}, and ω_{δi} in a case of Equation (1), and depends on the parameters αᵢ, γᵢ, δᵢ, N_{i*}, and ωᵢ in a case of Equation (2).

In addition, the parameter estimation unit 11 estimates parameters that minimize an error as defined below, by using cross validation and gradient descent.

$${SE}_{t e s t} = {\sum }_{( i , j , t ) ∈ t e s t} {( {r}_{ij} - 〈 {u}_{i} ( t ) , {v}_{j} 〉 )}^{2} \qquad [ Math . 8 ]$$

Examples of the output of the parameter estimation unit 11 are shown in Table 1 and Table 2.

TABLE 1

| | |
|---|---|
| Case of (1) | Case of (2) |

| User ID | αᵢ | γᵢ | δᵢ | ω_{γi} | ω_{δi} | Nᵢ* | ωᵢ |
|---|---|---|---|---|---|---|---|
| 1 | 0.3 | 0.4 | 0.4 | 0.3 | 0.6 | 0.2 | 0.5 |
| 2 | 0.5 | 0.2 | 0.3 | 0.6 | 0.2 | 0.7 | 0.3 |

TABLE 2

| ID of Behavior option | νⱼ from τ = 0 to τ = t − 1 |
|---|---|
| 1 | (0.2, 0.3, 0.5) |
| 2 | (0.7, 0.2, 0.1) |

The social welfare calculation unit 12 calculates a value indicating social welfare of the user, based on the estimated parameters and prediction target timing data 903. The social welfare of the user is expressed by the similarity between the tendency of the interest of the user and the type of the behavior that includes a plurality of factors.

The prediction target timing data 903 is data indicating a time at which the behavior for each user included in the behavior data 901 is evaluated. Values indicating respective behaviors, included in the behavior data 901, are arranged in time order. Thus, in the prediction target timing data 903, an elapsed time is represented in units of seconds, after the initial timing is set to 0 seconds.

Specifically, the social welfare calculation unit 12 calculates, for each user, a prediction value of the social welfare of the user, based on the estimated parameters and the prediction target timing data 903. The social welfare calculation unit 12 calculates, as a value indicating the social welfare, the inner product of uᵢ(t) and vⱼ, where uᵢ(t) is obtained by round-robin of possibilities of the tendency uᵢ(t) of the interest of the user, and vⱼ indicating a behavior option vⱼ.

The optimum behavior selection unit 13 selects, as an optimum behavior option, a behavior that maximizes the calculated value indicative of the social welfare of the user, and then outputs data (optimum behavior data 904) indicating the selected behavior option. Table 3 shows an example of the optimum behavior data 904.

TABLE 3

| | |
|---|---|
| | Behavior option with values (top three values) |
| User ID | indicating social welfare |

| | | | |
|---|---|---|---|
| 1 | ν₁₆ | ν₁₀₃ | ν₅₈ |
| 2 | ν₂₉₃ | ν₆₉ | ν₁₇₅ |

Table 3 shows the example in which behaviors of which values indicative of the social welfare are ranked at the top three are output as options. However, the scope of the present invention is not limited to the above example. The optimum behavior selection unit 13 may output data indicating a behavior of which a value indicative of the social welfare is ranked at the top, or may output data of behaviors that are ranked at the top two or the top four.

### (Operation of Information Processing Apparatus)

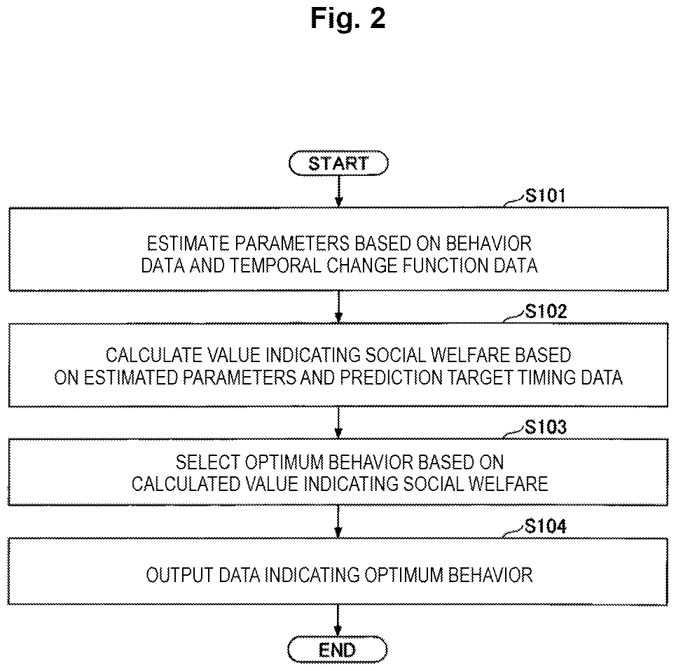

FIG. 2 is a flowchart illustrating an example of a flow of estimation processing. The information processing apparatus 10 performs estimation processing according to an operation of the user or the like. The parameter estimation unit 11 estimates parameters based on the behavior data 901 and the time-varying function data 902 (step S101).

Next, the social welfare calculation unit 12 calculates a value indicating social welfare, based on the estimated parameters and prediction target timing data 903 (step S102).

Subsequently, the optimum behavior selection unit 13 selects an optimum behavior based on the calculated value indicating the social welfare (step S103). In addition, the optimum behavior selection unit 13 outputs data (optimum behavior data 904) indicating the optimum behavior (step S104).

(Hardware Configuration Example according to Present Embodiment)

The information processing apparatus 10 can be implemented, for example, by a program that causes a computer to execute a process as described in the present embodiment. Note that the “computer” may include a physical machine, or may include a virtual machine in a cloud. When using a virtual machine, “hardware” described herein is virtual hardware.

The program can be stored and distributed by recording the program in a computer-readable recording medium (a portable memory or the like). Further, the program can be provided via a network such as the Internet or an electronic mail.

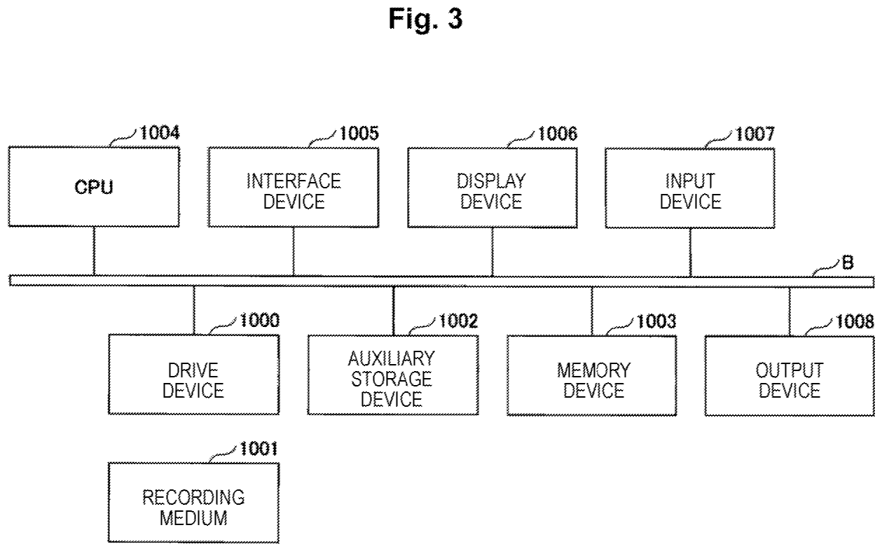

FIG. 3 is a diagram illustrating a hardware configuration example of the computer. The computer in FIG. 3 includes a drive device 1000, an auxiliary storage device 1002, a memory device 1003, a CPU 1004, an interface device 1005, a display device 1006, an input device 1007, an output device 1008, and the like, which are connected together via a bus B.

The program for implementing a process by the computer is provided by, for example, a recording medium 1001 such as a CD-ROM or a memory card. When the recording medium 1001 that stores the program is set in the drive device 1000, the program is installed from the recording medium 1001 into the auxiliary storage device 1002 via the drive device 1000. Here, the program is not necessarily installed from the recording medium 1001, and may be downloaded from another computer via a network. The auxiliary storage device 1002 stores the installed program, and also stores necessary files, data, and the like.

In response to receiving an instruction to start a program, the memory device 1003 reads the program from the auxiliary storage device 1002, and stores the program therein. The CPU 1004 implements a function related to the information processing apparatus, according to the program stored in the memory device 1003. The interface device 1005 is used as an interface for connecting to a network. The display device 1006 displays a graphical user interface (GUI) or the like according to the program. The input device 1007 includes a keyboard and a mouse, buttons, a touch panel, and the like, and is used to input various operation instructions. The output device 1008 outputs a calculation result.

In the information processing apparatus 10 according to the present embodiment, as a model for predicting social welfare of a user, a model obtained by introducing temporal changes of attraction and aversion for each user is used. Thereby, it is possible to accurately predict interest of the user, search for behavior options according to the interest of the user, and present a selection content that can maximally enhance social welfare. That is, u(t) according to the present embodiment is different from the function disclosed in Non-Patent Document 1 in that attraction and aversion change with time and dominance and recessiveness of attraction and aversion are gradually switched.

The parameter estimation unit 11 according to the present embodiment estimates parameters that minimize an error between the similarity between the tendency of the interest of the user and the type of the behavior including the plurality of elements and the evaluation for the behavior by using matrix decomposition. In particular, v(t) is a vector, and is expressed by a mix of interest in a plurality of elements such as romance and horror. Thus, it is possible to express complex preferences.

### SUMMARY OF EMBODIMENT

In this specification, at least an information processing apparatus, an estimation method, and a program described in each of sections to be described below are described.

### (First Item)

An information processing apparatus including: a parameter estimation unit that estimates, as parameters indicating a temporal change in interest of a user, a first parameter, a second parameter, and a third parameter based on data including a past behavior of the user and an evaluation value of the behavior, the first parameter being a parameter indicating inherent constant interest of the user, the second parameter being a parameter indicating an effect that the user is influenced by a past behavior and is attracted to a behavior option, and the third parameter being a parameter indicating an effect that the user loses interest due to boredom by a past behavior; social welfare calculation unit that calculates a value indicating social welfare of the user based on the estimated parameters; and an optimum behavior selection unit that selects an optimum behavior of the user based on the calculated value indicating social welfare and outputs data indicating the selected optimum behavior, in which the effect indicated by the second parameter is larger than the effect indicated by the third parameter in a case where a selection frequency of the behavior option is low in the past behaviors of the user, and is smaller than the effect indicated by the third parameter in a case where a selection frequency of the behavior option is high in the past behaviors of the user.

### (Second Item)

The information processing apparatus according to Section 1, in which the parameter estimation unit estimates, as parameters of a model that represents a temporal change in the interest of the user and calculates a weighted average with respect to a past behavior history by using weights which attenuate with time, the second parameter representing the weight and the third parameter which is different from the second parameter and represents the weight.

### (Third Item)

The information processing apparatus according to Section 1, in which the parameter estimation unit calculates parameters of a model representing a temporal change in the interest of the user based on a result obtained by calculating a similarity between a past behavior history and a latest behavior history.

### (Fourth Item)

The information processing apparatus according to any one of Sections 1 to 3, in which the parameter estimation unit estimates the parameters that minimize an error between a similarity between a tendency of the interest of the user and a type of the behavior including a plurality of elements and the evaluation for the behavior by using matrix decomposition.

### (Fifth Item)

An estimation method executed by a computer, the method including: a step of estimating, as parameters indicating a temporal change in interest of a user, a first parameter, a second parameter, and a third parameter based on data including a past behavior of the user and an evaluation value of the behavior, the first parameter being a parameter indicating inherent constant interest of the user, the second parameter being a parameter indicating an effect that the user is influenced by a past behavior and is attracted to a behavior option, and the third parameter being a parameter indicating an effect that the user loses interest due to boredom by a past behavior; a step of calculating a value indicating social welfare of the user based on the estimated parameters; and a step of selecting an optimum behavior of the user based on the calculated value indicating social welfare and outputting data indicating the selected optimum behavior, in which the effect indicated by the second parameter is larger than the effect indicated by the third parameter in a case where a selection frequency of the behavior option is low in the past behaviors of the user, and is smaller than the effect indicated by the third parameter in a case where a selection frequency of the behavior option is high in the past behaviors of the user.

### (Sixth Item)

A program for causing a computer to function as each unit of the information processing apparatus according to any one of Sections 1 to 4.

Although the present embodiment has been described above, the present invention is not limited to such a particular embodiment, and various modifications and changes can be made within the scope of the gist of the present invention described in the claims.

### REFERENCE SIGNS LIST

10 Information processing apparatus
11 Parameter estimation unit
12 Social welfare calculation unit
13 Optimum behavior selection unit
901 Behavior data
902 Time-varying function data
903 Prediction target time data
904 Optimum behavior data

1. An information processing apparatus comprising:
circuitry configured to:
estimate, as parameters indicating a temporal change in interest of a user, a first parameter, a second parameter, and a third parameter, based on data including a past behavior of the user and an evaluation value of the behavior, the first parameter indicating inherent constant interest of the user, the second parameter indicating a first effect of attracting a behavior option that is influenced by the past behavior, and the third parameter indicating a second effect of losing the interest due to boredom of the past behavior;
calculate a value indicating social welfare of the user, based on the estimated parameters;
select an optimum behavior of the user, based on the calculated value indicating the social welfare; and
output data indicating the selected optimum behavior,
wherein the first effect is greater than the second effect in a case where a selection frequency of the behavior option is lower than a threshold for past behaviors of the user, and
wherein the first effect is lower than the second effect in a case where the selection frequency of the behavior option is higher than the threshold for the past behaviors of the user.

2. The information processing apparatus according to claim 1, wherein the circuitry is configured to:
estimate, as parameters of a model that represents the temporal change in the interest of the user and that calculates a weighted average with respect to a past behavior history by using weights that are attenuated with time,
the second parameter affected by the weight, and
the third parameter different from the second parameter, the third parameter being affected by the weight.

3. The information processing apparatus according to claim 1, wherein the circuitry is configured to calculate parameters of a model representing a temporal change in the interest of the user, based on a result obtained by calculating a similarity between a past behavior history and a latest behavior history.

4. The information processing apparatus according to claim 1, wherein the circuitry is configured to use matrix decomposition to estimate the parameters that minimize an error between (i) a similarity between a tendency of the interest of the user and a type of the behavior including a plurality of factors and (ii) an evaluation for the behavior.

5. An estimation method executed by a computer, the method comprising:
estimating, as parameters indicating a temporal change in interest of a user, a first parameter, a second parameter, and a third parameter, based on data including a past behavior of the user and an evaluation value of the behavior, the first parameter indicating inherent constant interest of the user, the second parameter indicating a first effect of attracting a behavior option that is influenced by the past behavior, and the third parameter indicating a second effect of losing the interest due to boredom of the past behavior;
calculating a value indicating social welfare of the user based on the estimated parameters, and
selecting an optimum behavior of the user based on the calculated value indicating the social welfare; and
outputting data indicating the selected optimum behavior,
wherein the first effect is greater than the second effect in a case where a selection frequency of the behavior option is lower than a threshold for past behaviors of the user, and
wherein the first effect is lower than the second effect, in a case where the selection frequency of the behavior option is higher than the threshold for the past behaviors of the user.

6. A non-transitory computer readable medium storing a program for causing a computer to execute the estimation method of claim 5.
